(19) United States
(12) Patent Application Publication
(10) Pub. No.: US 2005/0107352 A1
(43) Pub. Date: May 19, 2005

(54) Method of treating benign prostatic hyperplasia

(76) Inventor: L. Dean Parks, Ocala, FL (US)

Correspondence Address: MELVIN K. SILVERMAN, 500 WEST CYPRESS CREEK ROAD, SUITE 500, FT. LAUDERDALE, FL, 33309, US

(21) Appl. No.: 10/980,570

(22) Filed: Nov. 2, 2004

Related U.S. Application Data
(63) Continuation-in-part of application No. 10/841,727, filed on May 6, 2004.
Application No. 10/841,727 is a continuation-in-part of application No. 09/953,620, filed on Sep. 17, 2001, now Pat. No. 6,733,779.
Application No. 09/953,620 is a continuation-in-part of application No. 09/383,608, filed on Aug. 26, 1999, now abandoned.

(51) Int. Cl. A61K 31/517; A61K 31/203

(52) U.S. Cl. 514/183; 514/252.16; 514/559; 514/252.17

(57) ABSTRACT

Methods of treatment of benign prostatic hyperplasia are disclosed. One method includes orally administering to a patient having benign prostatic hyperplasia an alpha-1 adrenoceptor blocking agent, and orally administering to the patient a therapeutically effective amount of 13-cis-retinoic acid. Another method includes orally administering to a BPH patient an inhibitor of steroid 5(alpha)-reductase, and orally administering to the patient a therapeutically effective amount of 13-cis-retinoic acid. The methods further include an initial treatment of patients with an initial dosage of about 0.25 mg per pound of body weight of 13 cis-retinoic acid daily for a period from about ten days to about twenty days, and followed by a sustaining treatment of the patients with a sustaining dosage of about 0.25 mg per pound of body weight of 13 cis-retinoic acid about every five to seven days in a sustaining period.

### CROSS REFERENCE TO RELATED APPLICATION

This application is a continuation-in-part application of patent application Ser. No. 10/841,727, filed May 6, 2004, which is a continuation-in-part application of patent application Ser. No. 09/953,620 filed Sep. 17, 2001, now U. S. Pat. No. 6,733,779, which is a continuation-in-part application of Ser. No. 09/383,608 filed Aug. 26, 1999, now abandoned. All prior patent applications are herein incorporated by reference in their entirety.

### FIELD OF THE INVENTION

The present invention relates to methods of treatment of benign prostate hyperplasia. More specifically, the methods utilize 13 cis-retinoic acid to effectively treat benign prostate hyperplasia.

### BACKGROUND OF THE INVENTION

The three most common prostate health problems facing men and their families today are benign prostatic hyperplasia (BPH), prostate cancer, and prostatitis. Each of these conditions affects the prostate differently. Benign prostate hyperplasia is the most common benign neoplasm (non cancerous enlargement of the prostate gland) in men, and has a high prevalence that increases with age. The increase in size of the prostate inside its capsule exerts pressure on the urethra, which passes through the capsule, resulting in obstruction to urine flow. Half of all men have BPH identifiable histologically at age 60 years, and by 85 years the prevalence is about 90%. In the United States about 25% of men will be treated for BPH by age 80, and over 300,000 surgical procedures are performed each year for BPH (mostly transurethral resection of the prostate, TURP). This makes TURP the second most common surgical procedure, second only to cataract surgery—at a cost estimated at $2 billion per year. Experts do not yet know what causes BPH, but the condition may be related to the hormone testosterone and its relationship to other hormones that change during the aging process.

There are a number of treatment options for BPH. These include watchful waiting, medical therapy such as alpha blocker therapy and finasteride therapy, balloon dilatation and various surgical procedures such as transurethral incision of the prostate (TUIP), transurethral resection of the prostate (TURP), and open prostatectomy. Few treatments are without any adverse consequences, and this is particularly so with treatments with BPH, where there is a delicate balancing act between the benefits and demerits of the treatments available. The adverse events following treatment for BPH include impotence (for various surgical procedures ranging from about 4% to 40%, the incidence of impotence is also increased after some medical treatments), incontinence (stress incontinence about 3% after surgery, with total urinary incontinence approaching 1%), and the need for re-treatment. Combined analysis of published data estimated that the mean probability for perioperative mortality (death within 90 days of a procedure) was 1.5% for TURP. For open surgery it was 2.4% and for balloon dilation it was 3.5%.

Currently, most commonly used hormone therapy is oral administration of finasteride. Finasteride, commercially available under the tradename Proscar® from Merck & Co. Inc., Whitehouse Station, N.J., is a synthetic 4-azasteroid compound, a specific inhibitor of steroid Type II 5α-reductase, and an intracellular enzyme that converts the androgen testosterone into 5α-dihydrotestosterone (DHT). Finasteride helps to shrink the enlarged prostate and reduces elevated PSA due to benign prostate conditions. However, finasteride is known to cause undesirable side effects, which includes impotence or less desire for sex, problems with ejaculation, and breast swelling enlargement and/or tenderness.

Alpha-1 adrenoceptor blocking agents are also currently used for clinical treatment of benign prostatic hyperplasia. Suitable examples include tamsulasin hydrochloride, terazosin hydrochloride, alfuzosin hydrochloride and doxazosin mesylate. The symptoms associated with benign prostatic hyperplasia are related to bladder outlet obstruction, which is comprised of two underlying components: a static component and a dynamic component. The static component is related to an increase in prostate size caused, in part, by a proliferation of smooth muscle cells in the prostatic strama. However, clinical studies have demonstrated that the size of the prostate does not always correlate with the severity of benign prostatic hyperplasia symptoms, or the degree of constriction of the bladder outlet. The dynamic component is a function of an increase in smooth muscle tone in the prostate and bladder neck, leading to constriction of the bladder outlet. Smooth muscle tone is mediated by the sympathetic nervous stimulation of alpha-1 adrenoceptors, which are abundant in the prostate, prostatic capsule, prostatic urethra, and bladder neck. The reduction in symptoms and improvement in urine flow rates following administration of an alpha-1 adrenoceptor blocking agent is related to relaxation of smooth muscle produced by blockage of alpha-1 adrenoceptors in the bladder neck and prostate.

Furthermore, plant sterols have been used for treating benign prostatic hyperplasia since 1970s.

It is well known that prostate specific antigen (PSA), a protein produced by prostate cells, is frequently present at elevated levels in the blood of men who have prostate cancer. The U.S. Food and Drug Administration has approved a PSA test for use in conjunction with a digital rectal exam to help detect prostate cancer in men age 50 or older and for monitoring prostate cancer patients after treatment. However, much remains unknown about the interpretation of PSA levels, the test's ability to discriminate cancer from benign prostate conditions, and the best course of action following a finding of elevated PSA. Furthermore, clinically it is known that BPH and prostatitis can cause elevated PSA. 13-cis retinoic acid, more generally known as retinoic acid, also referred to as isotretinoin, and sold under the Roche trademark Accutane®, has long been known as a topical and oral dermatologic agent used in the treatment of acne vulgrais and several other skin diseases. 13-cis retinoic acid inhibits sebaceous gland function and keratinization. The exact mechanism of action of Accutane® in treating acne is unknown. Since retinoic acid is a teratogenic drug and, because of the mutagenic effects associated with such drugs, it has only gradually entered the mainstream of medicine.

Experimentation in non-dermatologic applications of retinoic acid appeared in the literature in 1992 in an investigation at the University of California School of Medicine (Department of Urology), in San Francisco, this with reference to the effect of 13-cis-retinoic acid upon human prostate cancer cells. Since 1992, the research group at the University of California, headed by Dr. Dahiya, established the effect of retinoic acid in the downregulation of saturated fatty acids coupled with the upregulation of unsaturated fatty acids in human prostate cancer cells. As such, saturated fatty acids, which are believed to play a significant role in prostate cancer, were inhibited while unsaturated fatty acids, which are believed to act in a protective way relative to such cancers, were increased in cell lines.

However, isotretinoin or retinoic acid has not been used for treating benign prostate hyperplasia and related symptoms.

### SUMMARY OF THE INVENTION

In one embodiment, the present invention relates to a method of treating benign prostatic hyperplasia. The method comprises the steps of orally administering to a patient having benign prostatic hyperplasia a therapeutically effective amount of an alpha-1 adrenoceptor blocking agent, and orally administering to the patient a therapeutically effective amount of 13-cis-retinoic acid. The alpha-1 adrenoceptor blocking agent includes tamsulosin hydrochloride, terazosin hydrochloride, alfuzosin hydrochloride and doxazosin mesylate.

In a further embodiment, the method comprises the steps of orally administering to a patient having benign prostatic hyperplasia a therapeutically effective amount of an inhibitor of steroid 5(alpha)-reductase, and orally administering to the patient a therapeutically effective amount of 13-cis-retinoic acid. The inhibitor of steroid 5(alpha)-reductase includes finasteride and dutasteride.

In another embodiment, the method comprises using a therapeutically effective amount of 13-cis-retinoic acid together with plant sterols.

Preferably, the method comprises orally administering to the patient an initial dosage of about 0.25 mg per pound of body weight of 13-cis-retinoic acid for an initial treatment period, and thereafter orally administering a sustaining dosage of about 0.25 mg per pound of body weight of 13-cis-retinoic acid about every five to seven days in a sustaining period.

It is accordingly an object of the present invention to provide a method of treating benign prostatic hyperplasia and related symptoms such as reduced or constricted urine stream and urinary retention.

It is another object to provide an enhanced treatment method which combines the traditional treatment using alpha-1 adrenoceptor blocking agent, inhibitor of steroid 5(alpha)-reductase or plant sterols with the use of 13-cis retinoic acid.

The above and yet other objects and advantages of the present invention will become apparent from the hereinafter set forth Detailed Description of the Invention and Claims appended herewith.

### DETAILED DESCRIPTION OF THE INVENTION

In one embodiment, the present invention provides methods of treatment of benign prostatic hyperplasia and reduction of the level of the prostate specific antigen (PSA) of benign prostate conditions. The instant invention derived from the observation that the human prostate comprises a modified sebaceous gland and that, given the many years of usage of successful 13-cis-retinoic acid in the treatment of acne, this through the shrinking of sebaceous glands within the face and neck, that a similar action might occur upon the prostate gland with oral administration of 13-cis-retinoic acid.

Operating with the informed consent of approximately twelve patients exhibiting either or both an elevated PSA, that is, patients with a PSA of greater than 4 ng/ml and/or with exhibiting benign prostatic hyperplasia, the patients were treated with Accutane® manufactured by Roche.

The treatment method involves essentially two periods: an initial treatment, and a sustaining treatment. First, in the initial treatment the patients orally administrate an initial dosage about 40 mg of 13-cis-retinoic acid daily for a period from about ten days to about twenty days. Thereafter, the patients orally administrate a sustaining dosage about 40 mg of 13-cis-retinoic acid about every five to seven days in a sustaining period. Typically, the sustaining period is about one year. However, it can be extended longer if extended maintenance is needed. Preferably, the initial treatment period is about fourteen days, because beyond fourteen days side effects of the medicine, such as tenderness at sites of old injuries to the fibro-musculo-skeletal system, dry skin, chapped lips, dry eyes, and dry nose, tend to occur. If these side effects occur during the initial treatment period, they normally subside after the sustaining dosage is instituted.

In the initial treatment, 40 mg of 13-cis-retinoic acid can be administrated in a single dose, or divided doses. Commercially, Accutane® has three available doses, 10 mg, 20 mg, and 40 mg soft gelatin capsules. The patients can either take one 40 mg capsule, or take two 20 mg capsules daily. The same applies to the dosage in the sustaining treatment. For convenience, the patients can take one 40 mg dose every five to seven days.

The initial dosage of about 40 mg 13-cis-retinoic acid daily is determined based on average men's body weight of about 160 lbs, i.e. at about 0.25 mg/lb of body weight. For patients who weigh substantially above or less than the average body weight, the dosage described above can be adjusted accordingly. The same principle applies to the sustaining dosage.

The 13-cis-retinoic acid used for the purpose of the present invention can be in various forms, such as powders, pills, capsules, tablets, and liquids. It is known that the commercially available product Accutane® in the form of capsules contains various pharmaceutically acceptable media including beewax, butylated hydroxyanisole, edetate disodium, hydrogenated soybean oil flakes, hydrogenated vegetable oil, glycerin. The tablets can contain pharmaceutically acceptable excipients which are suitable for the manufacture of tablets. Such excipients include, for example, inert diluents, such as calcium phosphate, calcium carbonate, sodium carbonate, sodium phosphate, or lactose; granulating disintegrating agents, for example, maize starch or alginic acid; binding agents, such as starch, gelatin, or acacia; and lubricating agents, for example, magnesium stearate, stearic acids or talc. Compressed tablets may be uncoated or may be sugar coated or film coated by known techniques to mask any unpleasant taste and protect the tablet from the atmosphere, or enteric coated for selective disintegration and adsorption in the gastrointestinal tract. Hard gelatin capsules or liquid filled soft gelatin capsules contain the active ingredient and inert powdered or liquid carriers, such as, but not limited to calcium carbonate, calcium phosphate, kaolin, lactose, lecithin starch, cellulose derivatives, magnesium stearate, stearic acid, arachis oil, liquid paraffin, olive oil, pharmaceutically-accepted synthetic oils and other diluents suitable for the manufacture of capsules. Both tablets and capsules can be manufactured as sustained release-products to provide for continuous release of medication over a period of hours. The media or excipients suitable for the manufacture of aqueous suspensions include suspending agents, e.g., maltodextrin, sodium carboxymethylcellulose, methylcellulose, hydroxypropylmethylcellulose, sodium alginate, polyvinylpyrrolidone, gum tragacanth, and gum acacia; dispersing or wetting agents, such as a naturally occurring phosphatide, e.g., lecithin, or condensation products of an alkylene oxide with fatty acids, for example of polyoxyethylene stearate, or a condensation products of ethylene oxide with long chain aliphatic alcohols, e.g., heptadecaethyleneoxycetanol, or condensation products of ethylene oxide with partial esters derived from fatty acids and a hexitol, e.g., polyoxyethylene sorbitol monooleate, or a condensation product of ethylene oxide with partial esters derived from fatty acids and hexitol anhydrides, e.g., polyoxyethylene sorbitan monooleate. The aqueous suspensions can also contain one or more preservatives, for example ethyl, n-propyl, or p-hydroxy benzoate, one or more coloring agents, one or more flavoring agents, and one or more sweetening agents, such as sucrose, saccharin, or sodium or calcium cyclamate. Other suitable media include water, glycol, oil, alcohol and syrup.

This treatment method resulted in subjective as well as objective amelioration of benign prostatic hyperplasia and symptoms thereof, and a measurable reduction in the prostate specific antigen (PSA). In particular, through examination of these patients a reduction in prostate enlargement was confirmed. Furthermore, the blood test results showed that PSA levels had been substantially reduced to levels in a range of 0 to 2 ng/ml among these patients.

In a further embodiment, the present invention provides a method of treatment of benign prostatic hyperplasia using a combination of traditional hormone treatment and the 13-cis-retinoic acid treatment described above.

The term of “traditional hormone treatment” means the hormone treatments known in the art, which are currently used clinically to treat benign prostatic hyperplasia. Suitable examples include oral administration of finasteride or dutasteride. Finasteride, available under the tradename Proscar® from Merck & Co. Inc., as a hormone treatment alone, is currently used for six month with a recommend dosage of 5 mg orally once daily. Dutasteride, available under the tradename Avodart® from GlaxoSmithKline, Research Triangle Park, N.C., is a synthetic 4-azasteroid compound. The recommended dosage of dutasteride for treating benign prostatic hyperplasia is 0.5 mg orally once daily. Both finasteride and dutasteride are inhibitor of both type 1 and type 2 isoforms of steroid 5(alpha)-reducatase, an intracellular enzyme that converts testosterone to 5(alpha)-dihydrotestosterone (DHT). DHT is the androgen primarily responsible for the initial development and subsequent enlargement of the prostate gland.

With the combinational treatment, two approaches can be taken. The first is to maintain the regular dosage of finasteride, or dutasteride and to orally administer 13-cis retinoic acid with a dosage of about 0.25 mg per pound of body weight to enhance the effect of treatment. The second is for the patient who has side effect or low tolerance of hormone treatment. In this approach, the dosage of 13-cis retinoic acid is the same as in the first approach, but the dosage of finasteride or dutasteride can be reduced. For example, finasteride can be reduced to a range from about 1 mg to about 3 mg daily, which can be determined according to the patient's response. Furthermore, with the combinational treatment, the dosage of finasteride can further be reduced to from about 1 mg to about 3 mg every other day. In either approach, it is preferred that the administration of 13-cis retinoic acid includes using the initial dosage in the initial treatment period and sustaining dosage in the sustaining treatment period, as described hereinbefore.

In another embodiment, the present invention provides a method of treatment of benign prostatic hyperplasia using a combination of alpha-1 adrenoceptor blocking agent and the 13-cis-retinoic acid treatment described above. As described previously, alpha-1 adrenoceptor blocking agents are currently used for clinical treatment of benign prostatic hyperplasia, based on the mechanism of relaxing smooth muscle produced by blockage of alpha-1 adrenoceptors in the bladder neck and prostate. Suitable examples of alpha-1 adrenoceptor blocking agents can be used for the combinational treatment of the present invention include, but are not limited to, tamsulasin hydrochloride, terazosin hydrochloride, alfuzosin hydrochloride and doxazosin mesylate.

One preferred alpha-1 adrenoceptor blocking agent is tamsulasin hydrochloride which is available under the tradename Flomax® from Boehringer Ingelheim Pharmaceuticals, Inc. Ridgefield, Conn. Recommended dosage of tamsulasin hydrochloride is 0.4 mg once daily for treatment of the symptoms of benign prostatic hyperplasia.

Another preferred alpha-1 adrenoceptor blocking agent is terazosin hydrochloride, which is available under the tradename Hytrin® from Abbott Laboratories, North Chicago, Ill. Hytrin® capsules for oral ingestion are available in four dosage strengths containing 1 mg, 2 mg, 5 mg, or 10 mg of terazosin hydrochloride. The recommended dosage for treating BPH is 1 mg at bedtime as the initial dose, and subsequently increasing in a stepwise fashion to 2 mg, 5 mg, or 10 mg once daily to achieve the desired improvement of symptoms and/or flow rates. When used alone, dosage of 10 mg once daily are generally required for the clinical response. In the combinational treatment of the present invention, the treatment process involving an initial dosage and a subsequent dosage of terazosin hydrochloride is maintained. However, the subsequent dosage itself can be varied, preferably reduced, depending on the patient's response to the combinational treatment.

Alfuzosin hydrochloride is available in extended-release tablets under the tradename Uroxatral® from Sanofi-Synthelabo Inc., New York, N.Y. The recommended dosage for treatment of benign prostatic hyperplasia is one Uroxatral tablet containing 10 mg alfuzosin hydrochloride daily. Doxazosin mesylate is available in tablets under the tradename Cardura® from Pfizer Roerig, Division of Pfizer Inc., New York, N.Y. The recommended dosage for treatment of benign prostatic hyperplasia is 1 to 8 mg doxazosin mesylate once daily. The initial dosage of Cardura® is 1 mg once daily. The dosage may then be increased to 2 mg and thereafter to 4 mg and 8 mg once daily, depending on the individual patient's urodynamics and BPH symptomatology.

Similar to the combinational treatment with the hormone treatment, the dosage of alpha-1 adrenoceptor blocking agents can either be maintained as recommended in its individual use, or reduced because of the synergetic effect obtained from two independent treatment mechanisms. The latter can be particularly beneficial for the patients who experience side effects of the alpha-1 adrenoceptor blocking agent. Again in either approach, it is preferred that the administration of 13-cis retinoic acid includes using the initial dosage in the initial treatment period and sustaining dosage in the sustaining treatment period, as described hereinbefore.

In yet a further embodiment, 13-cis retinoic acid can also be used in conjunction with oral administration of plant sterols for treating benign prostatic hyperplasia. Plant sterols or phyto-sterols contain β-sitosterol and its glycoside, β-sitosterolin, which can be found in the normal diet. β-sitosterol has a modulating effect in vitro on prostatic growth factor (beta 1) and the activity of a protein kinase (Cα) in primary prostate stromal cultures. As reported in clinical trials using plant sterols for treating benign prostatic hyperplasia, there were significant improvements in symptoms and urine flow dynamics in the trials, although no reduction of prostate size. The plant sterol dietary supplement is commercially available from various sources. Suitable examples are the commercial products under the trade names of Harzol® from Hoyer&Co., Germany, and Moducare® from Essential Phytosterolins, Inc. Moducare® is a blend of plant sterols and sterolins, and the recommended dosage is 3 capsules daily. The combination of plant sterols with the method of the present invention using 13-cis retinoic acid can further enhance the effect of treating benign prostatic hyperplasia.

Examples 1 to 4 illustrate clinical effectiveness of the above described treatment method. It was found that none of the conditions of these patients worsened either in terms of PSA or hyperplasia, and none progressed into any form of diagnosable malignancy. Further, no serious adverse side effects or contra-indications were observed among the patients in the informal trials.

### EXAMPLE 1

A patient started a slowly progressive prostatic enlargement and attenuated urine stream 20 years ago. Hand examination confirmed a palpably enlarged prostate, probably secondary to post vasectomy prostatitis.

The patient was placed on 40 mg of 13-cis retinoic acid (in the form of Accutane®) orally for 14 days in the initial treatment. Then he was placed on sustaining dose of 40 mg of 13-cis retinoic acid (in the form of Accutane®) every 5 days for about one year.

The patient's symptoms improved steadily. His prostate decreased in palpable size, and PSA dropped from 4 to less than one.

No adverse side effects were noted other than drying of lips and some musculo-skeletal soreness at sites of old injuries to the fingers and neck. This soreness subsided slowly after the sustaining dose was instituted. Blood chemistries remained normal on this low sustaining dosage.

### EXAMPLE 2

The patient had prior histories of urethritis and prostatitis from sexually transmitted diseases. The patient had steadily increasing prostatic enlargement, with decreasing urine flow, plus urgency and nocturia. PSA was 3, and prostate palpably enlarged.

The patient was placed on 40 mg of 13-cis retinoic acid (in the form of Accutane®) orally for 14 days. Then the dosage was decreased to 40 mg of 13-cis retinoic acid (in the form of Accutane®) every 5 days for about one year.

The patient had steady improvement in symptoms and after 6 months PSA had dropped to less than one. No adverse side effects were observed from administration of the Accutane®.

### EXAMPLE 3

A patient had history of post-traumatic prostatitis caused by a bicycle accident in 1987. The patient's condition became chronic especially after long auto trips, and required frequent courses of antibiotics and steroids to calm the inflammation. PSA was 5.

The patient was placed on 40 mg of 13-cis retinoic acid (in the form of Accutane®) daily for 14 days. Thereafter, the patient had a maintenance dosage of 40 mg of 13-cis retinoic acid (in the form of Accutane®) every 5 days for about one year.

The patient rapidly became symptom free after the initial treatment, and had remained so during the sustaining period. PSA dropped to less than 1.

### EXAMPLE 4

The patient had a history of sexually transmitted diseases in college and subsequent history of prostatitis. Enlargement of prostate was noted 6 years prior to the Accutane treatment, with steadily worsening urinary tenesmus.

The patient was placed on 40 mg of 13-cis retinoic acid (in the form of Accutane®) per day. After 14 days, the dosage was decreased to 40 mg of 13-cis retinoic acid (in the form of Accutane®) every 5 days for about one year.

The patient's symptoms readily improved and PSA dropped from 4 to less than 1 within 6 months.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith.

1. A method for treating benign prostatic hyperplasia comprising the steps of:
(a) orally administering to a patient having benign prostatic hyperplasia a therapeutically effective amount of an alpha-1 adrenoceptor blocking agent, and
(b) orally administering to said patient a therapeutically effective amount of 13-cis-retinoic acid.

2. The method of claim 1, wherein said alpha-1 adrenoceptor blocking agent is tamsulosin hydrochloride.

3. The method of claim 1, wherein said alpha-1 adrenoceptor blocking agent is terazosin hydrochloride.

4. The method of claim 1, wherein said alpha-1 adrenoceptor blocking agent is alfuzosin hydrochloride.

5. The method of claim 1, wherein said alpha-1 adrenoceptor blocking agent is doxazosin mesylate.

6. The method of claim 1, wherein step (b) further comprises orally administering to said patient an initial dosage of about 0.25 mg per pound of body weight of 13-cis-retinoic acid for an initial treatment period, and thereafter orally administering a sustaining dosage of about 0.25 mg per pound of body weight of 13-cis-retinoic acid about every five to seven days in a sustaining period.

7. The method of claim 6, wherein said initial treatment period is from about ten days to about twenty days.

8. The method of claim 7, wherein said sustaining period is about one year.

9. The method of claim 1, wherein said 13-cis-retinoic acid is in a pharmaceutical composition comprising a pharmaceutical acceptable medium.

10. The method of claim 9, wherein said pharmaceutical composition is in a form selected from the group consisting of powder, pill, capsule, tablet and liquid.

11. A method for treating benign prostatic hyperplasia comprising the steps of:
(a) orally administering to a patient having benign prostatic hyperplasia a therapeutically effective amount of an inhibitor of steroid 5(alpha)-reductase, and
(b) orally administering to said patient a therapeutically effective amount of 13-cis-retinoic acid.

12. The method of claim 11, wherein said inhibitor of steroid 5(alpha)-reductase is finasteride.

13. The method of claim 11, wherein said inhibitor of steroid 5(alpha)-reductase is dutasteride.

14. The method of claim 11, wherein step (b) further comprises orally administering to said patient an initial dosage of about 0.25 mg per pound of body weight of 13-cis-retinoic acid for an initial treatment period, and thereafter orally administering a sustaining dosage of about 0.25 mg per pound of body weight of 13-cis-retinoic acid about every five to seven days in a sustaining period.

15. The method of claim 14, wherein said initial treatment period is from about ten days to about twenty days.

16. The method of claim 15, wherein said sustaining period is about one year.

17. The method of claim 11, wherein said 13-cis-retinoic acid is in a pharmaceutical composition comprising a pharmaceutical acceptable medium.

18. The method of claim 17, wherein said pharmaceutical composition is in a form selected from the group consisting of powder, pill, capsule, tablet and liquid.

19. A method for treating benign prostatic hyperplasia comprising the steps of:
(a) orally administering to a patient having benign prostatic hyperplasia a therapeutically effective amount of 13-cis-retinoic acid; and
(a) orally administering to said patient a therapeutically effective amount of plant sterols.
